(19) United States
(12) Patent Application Publication
(10) Pub. No.: US 2008/0070860 A1
(43) Pub. Date: Mar. 20, 2008

(54) Adenosine Analogs Useful as Anti-Bacterial and Anti Protozoan Agents

(75) Inventors: Floyd Frederick Snyder, Calgary (CA); Thomas George Back, Calgary (CA)

Correspondence Address: BENNETT JONES;C/O MS ROSEANN CALDWELL, 4500 BANKERS HALL EAST, 855 - 2ND STREET, SW, CALGARY, AB, T2P 4K7, CA

(73) Assignee: UTI LIMITED PARTNERSHIP, Calgary, AB (CA)

(21) Appl. No.: 11/814,519

(22) PCT Filed: Feb. 3, 2006
(86) PCT No.: PCT/CA06/00140

§ 371 Date: Sep. 18, 2007

Related U.S. Application Data
(60) Provisional application No. 60/593,678, filed on Feb. 4, 2005.

(51) Int. Cl. A61K 31/7076; A61P 33/02; C07H 19/16

(52) U.S. Cl. 514/46; 536/28.8

(57) ABSTRACT

The present invention is directed to purine nucleoside analogs of the general Formula (I), or tautomers thereof, physiologically acceptable salts, solvents and physiologically functional derivatives thereof, and pharmaceutical compositions comprising such compounds, salts and derivatives, which are useful as anti-bacterial and anti-protozoan agents. The invention is also directed to methods for treating a bacterial or protozoan infection in a mammal and use of the compounds for inhibiting the growth of a bacteria or protozoa.

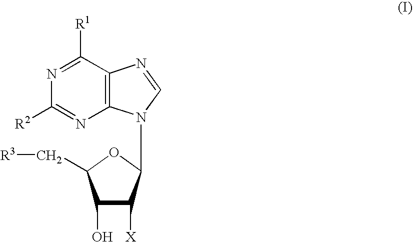

This application claims the benefit of U.S. Provisional Application No. 60/593,678, filed Feb. 4, 2005.

### FIELD OF USE

The present invention relates to purine nucleoside analogs useful as anti-bacterial and anti-protozoan agents. More particularly, the present invention relates to novel adenosine analogs, the use of these compounds as pharmaceuticals, pharmaceutical compositions containing the compounds and processes for preparing the compounds.

### BACKGROUND OF THE INVENTION

Infectious diseases remain a serious global health problem with significant rates of morbidity and mortality, especially in the young and in the elderly. In 1998, according to the World Health Organization, infectious diseases claimed 16 million lives and ranked as the world's second leading cause of death. There has been a resurgence of long-time killers such as tuberculosis, and the emergence of antibiotic-resistant strains of several key pathogens. Of particular concern is the increase in nosocomial infections, with associated high rates of morbidity and mortality (up to 50% in pneumonia and septicaemia). Furthermore, rampant and uncontrolled tropical protozoan diseases, such as malaria, Leishmaniasis, and Chagas' disease, affect mainly Southeastern Asia, Sub-Sahara Africa, and Latin America. The estimated number of cases is 350 million and annual number of deaths is 1.5 million. The need for novel classes of anti-bacterial and anti-protozoan agents is clear and urgent.

There is also an increased and widespread prevalence of microbial antibiotic resistance. For example, reports of methicillin-resistant Staphylococcus aureus (MRSA) with reduced susceptibility to vancomycin, the drug of choice for the treatment of MRSA, have been documented in the USA, Europe, and Japan. Even the oxazolidinone, linezolid (Pharmacia), which is the first new class of antibiotics to be introduced in the past 30 years and which was approved in 2000 for use in treating vancomycin-resistant Enterococcus faecalis (VRE) and MRSA, was met by resistance within one year of introduction.

Of particular concern is the increase in hospital-acquired (nosocomial) infections, with an incidence of 10 per 1000 patient days in OECD countries and with at least 70% of all infections involving antibiotic-resistant strains. For example, Pseudomonas aeruginosa, MRSA, and VRE account for 34% of all nosocomial infections. Another major concern is the prevalence (approaching 40%) of drug-resistant Streptococcus pneumoniae (DRSP) in community-acquired infections (mainly pneumonia but also otitis media and meningitis).

For most protozoan parasitic diseases, such as Cryptosporidiosis, Giardiasis, Malaria, Leishmaniasis, and Chagas' disease, there is a paucity of safe and efficacious drugs, and once-effective drugs are becoming obsolete due to the emergence of resistant strains. Expert panels (see, for example, Science, vol. 297, Jul. 19, 2002, pp. 343-344) have expressed a need for 20-30 new drugs to control protozoan diseases rampant in the tropics. In developed countries (OECD members), the incidence of parasitic disease is largely due to travelers to developing countries, with the exception of sporadic waterborne outbreaks of Cryptosporidiosis and Giardiasis due to failures in water treatment facilities.

The present invention looks at the use of novel purine nucleoside analogs as anti-bacterial and anti-protozoan agents. Both bacteria and protozoa are capable of synthesizing purine nucleotides through salvage pathways from preformed purine nucleosides. There are significant adaptive and energy savings in having the capacity to directly salvage purine nucleosides. Exogenous and endogenous nucleosides are utilized through two main salvage pathways. One of the salvage pathways involves enzymes having adenosine phosphorylase activities for the conversion of adenosine and deoxyadenosine to the free base adenine and the corresponding sugar moiety. Both bacteria (see, for example, Stoexkler, J. D., Agarwal, R. P., Agarwal, K. C., Schmid, K. and Parks, Jr., R. E. (1978) Biochemistry 17, 278-283; and Mao, C., Cook, W. J., Zhou, M., Koxzalka, G. W., Krenitsky, T. A. and Earlick, S. E. (1997) Structure 5, 1373-1383) and protozoa (see, for example, Bzowska A, Kulikowska E., and Shugar D., Biochim Biophys Acta (1992) 1120, 239-247; Trembacz, H., and Jezewska M. M., Adv Exp Med Biol (1998) 431, 711-717; Trembacz, H., and Jezewska, M. M., Comp Biochem Physiol B (1993) 104, 481-487; Dovey, H. F., McKerrow, J. H. and Wang C. C., Mol Biochem Parasitol (1985) 16, 185-198; Barankeiwicz J., and Jezewska M. M., Comp Biochem Physiol B (1976) 54, 239-242; Guranowski, A., and Wasternack C., Comp Biochem Physiol B (1982) 71, 483-488; Miech F. P., Senft A. W., and Senft D. G., Biochem Pharmacol (1975) 24, 407-411; and Munagala N. and Wang C. C., Biochemistry (2002) 41, 10382-10389) encode and express adenosine phosphorylase (AP) activity.

Mammals lack a comparable AP activity. The ubiquitous mammalian enzyme purine nucleoside phosphorylase (PNP) catalyzes the conversion of inosine or guanosine nucleosides to their respective bases, hypoxanthine or guanine, and ribose-phosphate, but does not act on adenosine (see Krenitsky, T. A., Elion, G. B., Henderson, A. M. and Hitchings, G. H., (1968) J. Biol. Chem. 243, 2867-2881 and Stoeckler, J. D., Agarwal, R. P., Agarwal, K. C., Schmid, K. and Parks, Jr., R. E., (1978) Biochemistry 17, 278-283). Therefore, analogs of adenosine, which can be acted upon by bacterial or protozoan AP but not mammalian PNP could potentially be useful agents in the treatment of bacterial or protozoan infections.

Preferably, adenosine analogs may also be refractory to other mammalian enzymes. In particular, adenosine analogs may be refractory to direct phosphorylation via adenosine kinase, deoxycytidine kinase and deoxyadenosine kinase, or deamination and removal via adenosine deaminase. Modification in the 5′-nucleoside position of adenosine is the most efficient approach to generating analogs refractory to phosphorylation. Successful modification of the 5′-nucleoside position, for example, to yield 5′-deoxy-5′-amino-adenosine, has been taught in the reference Kowaluk, E. A., Bhagwat, S. S. and Jarvis, M. F., Curr Pharm Des (1998) 5, 403-416. The analog 5′-deoxy-5′-amino-adenosine has been shown to act as a potent inhibitor of adenosine kinase. Substitutions in the 2-position of the purine ring significantly reduce rates of deamination via adenosine deaminase, a reaction that would generally remove the compound from being a useful pro-drug. In addition, moieties other than a 6-amino group, such as 6-methyl, would not act as substrates for adenosine deaminase.

### SUMMARY OF THE INVENTION

The present invention relates to purine nucleoside analogs that are effective anti-bacterial and anti-protozoan agents. More particularly, the invention features purine nucleoside analogs that are selective ligands of the purine salvage pathway enzyme adenosine phosphorylase (AP) found in bacteria and protozoa.

In one aspect of the present invention, compounds of Formula (I) are provided: 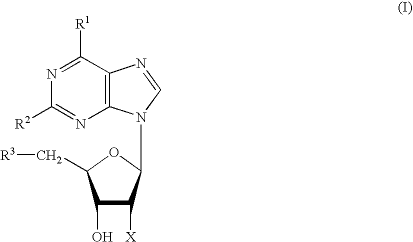
wherein:
R¹ is an amino, lower alkyl, sulfhydryl, lower alkylthio or lower alkoxy;
R² is a halogen, amino, hydrogen or lower alkyl;
R³ is an amino, lower alkoxy, lower alkyl or hydrogen; and
X is a hydroxy or hydrogen;
provided that:
(a) when X is hydroxy, R² is fluoro and R¹ is amino then R³ is not hydrogen;
(b) when X is hydroxy and R¹ is methyl, R² and R³ are not both hydrogen;
(c) when X is hydroxy, R² is chloro and R³ is methoxy, R¹ is not amino; and
(d) when X is hydroxy, R¹ is sulfhydryl and R² is hydrogen, R³ is not hydrogen;
or a physiologically acceptable salt or solvates thereof.

Preferred compounds of Formula (I) of the invention include those compounds where R¹ is an amino, methyl, sulfhydryl or methylthio group; R² is a chloro, fluoro, amino group or hydrogen; and R³ is hydrogen, methoxy or amino group; provided that when X is hydroxy, R² is fluoro and R¹ is an amino group, R³ is not hydrogen; when X is hydroxy and R¹ is methyl, R² and R³ are not both hydrogen; when X is hydroxy, R² is chloro and R³ is methoxy, R¹ is not amino; and when X is hydroxy, R¹ is sulfhydryl and R² is hydrogen, R³ is not hydrogen; or a physiologically acceptable salt or solvates thereof.

Particularly preferred compounds of Formula (I) of the invention include:
(1) 2-chloro-5′-deoxyadenosine;
(2) 2-chloro-6-methylpurine-5′-deoxyriboside;
(3) 2-chloro-6-mercaptopurine-5′-deoxyriboside;
(4) 5′-deoxyadenosine;
(5) 2-fluoro-6-methylpurine-5′-deoxyriboside;
(6) 2-amino-6-methylpurine-5′-deoxyriboside;
(7) 2-fluoro-6-mercaptopurine-5′-deoxyriboside;
(8) 2-amino-6-mercaptopurine-5′-deoxyriboside;
(9) 6-methylthiopurine-5′-deoxyriboside;
(10) 2-chloro-6-methylthiopurine-5′-deoxyriboside;
(1 1) 2-fluoro-6-methylthiopurine-5′-deoxyriboside;
(12) 2-amino-6-methylthiopurine-5′-deoxyriboside;
(13) 2-fluoro-5′-O-methyladenosine;
(14) 6-methylpurine-5′-O-methylriboside;
(15) 2-amino-5′-O-methyladenosine;
(16) 6-mercaptopurine-5′-O-methylriboside;
(17) 2-chloro-6-methylpurine-5′-O-methylriboside;
(18) 2-chloro-6-mercaptopurine-5′-O-methylriboside;
(19) 5′-O-methyladenosine;
(20) 2-fluoro-6-methylpurine-5′-O-methyl riboside;
(21) 2-amino-6-methylpurine-5′-O-methylriboside;
(22) 2-fluoro-6-mercaptopurine-5′-O-methylriboside;
(23) 2-amino-6-mercaptopurine-5′-O-methylriboside;
(24) 6-methylthiopurine-5′-O-methylriboside;
(25) 2-chloro-6-methylthiopurine-5′-O-methylriboside;
(26) 2-fluoro-6-methylthiopurine-5′-O-methylriboside;
(27) 2-amino-6-methylthiopurine-5′-O-methylriboside;
(28) 2-chloro-5′-aminodeoxyadenosine;
(29) 2-fluoro-5′-aminodeoxyadenosine;
(30) 6-methylpurine-5′-aminodeoxyriboside;
(31) 2-amino-5′-aminodeoxyadenosine;
(32) 6-mercaptopurine-5′-aminodeoxyriboside;
(33) 2-chloro-6-methylpurine-5′-aminodeoxyriboside;
(34) 2-chloro-6-mercaptopurine-5′-aminodeoxyriboside;
(35) 5′-aminodeoxyadenosine;
(36) 2-fluoro-6-methylpurine-5′-aminodeoxyriboside;
(37) 2-amino-6-methylpurine-5′-aminodeoxyriboside;
(38) 2-fluoro-6-mercaptopurine-5′-aminodeoxyriboside;
(39) 2-amino-6-mercaptopurine-5′-aminodeoxyriboside;
(40) 6-methylthiopurine-5′-aminodeoxyriboside;
(41) 2-chloro-6-methylthiopurine-5′-aminodeoxyriboside;
(42) 2-fluoro-6-methylthiopurine-5′-aminodeoxyriboside; and
(43) 2-amino-6-methylthiopurine-5′-aminodeoxyriboside.

More particularly preferred compounds of Formula (I) include 2-chloro-5′-deoxyadenosine, 2-chloro-6-methylpurine-5′-deoxy-β-D-riboside, 2-chloro-6-mercaptopurine-5′-deoxy-β-D-riboside and 2-fluoro-5′-O-methyladenosine.

According to a further aspect, the present invention provides a method of treating bacterial or protozoan infections which comprises administering to a mammal (including a human) suffering from infection with a bacteria or protozoa a therapeutically effective amount of a compound of Formula (I): 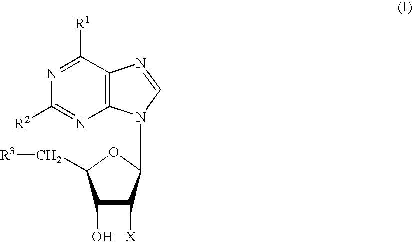
wherein:
R¹ is an amino, lower alkyl, sulfhydryl, lower alkylthio, or lower alkoxy;
R² is a halogen, amino, hydrogen or lower alkyl;
R³ is an amino, lower alkoxy, lower alkyl or hydrogen; and
X is a hydroxy or hydrogen;
or a physiologically acceptable salt or solvates thereof.

Preferred compounds of Formula (I) for treating bacterial or protozoan infections include those compounds where R¹ is an amino, methyl, sulfhydryl or methylthio group; R² is a chloro, fluoro, amino group or hydrogen; and R³ is hydrogen, methoxy or an amino group. Particularly preferred compounds of Formula (I) for treating bacterial or protozoan infections include:
(1) 2-chloro-5′-deoxyadenosine;
(2) 2-chloro-6-methylpurine-5′-deoxyriboside;
(3) 2-chloro-6-mercaptopurine-5′-deoxyriboside;
(4) 5′-deoxyadenosine;
(5) 2-fluoro-6-methylpurine-5′-deoxyriboside;
(6) 2-amino-6-methylpurine-5′-deoxyriboside;
(7) 2-fluoro-6-mercaptopurine-5′-deoxyriboside;
(8) 2-amino-6-mercaptopurine-5′-deoxyriboside;
(9) 6-methylthiopurine-5′-deoxyriboside;
(10) 2-chloro-6-methylthiopurine-5′-deoxyriboside;
(11) 2-fluoro-6-methylthiopurine-5′-deoxyriboside;
(12) 2-amino-6-methylthiopurine-5′-deoxyriboside;
(13) 2-fluoro-5′-O-methyladenosine;
(14) 6-methylpurine-5′-O-methylriboside;
(15) 2-amino-5′-O-methyladenosine;
(16) 6-mercaptopurine-5′-O-methylriboside;
(17) 2-chloro-6-methylpurine-5′-O-methylriboside;
(18) 2-chloro-6-mercaptopurine-5′-O-methylriboside;
(19) 5′-O-methyladenosine;
(20) 2-fluoro-6-methylpurine-5′-O-methylriboside;
(21) 2-amino-6-methylpurine-5′-O-methylriboside;
(22) 2-fluoro-6-mercaptopurine-5′-O-methylriboside;
(23) 2-amino-6-mercaptopurine-5′-O-methylriboside;
(24) 6-methylthiopurine-5′-O-methylriboside;
(25) 2-chloro-6-methylthiopurine-5′-O-methylriboside;
(26) 2-fluoro-6-methylthiopurine-5′-O-methylriboside;
(27) 2-amino-6-methylthiopurine-5′-O-methyl riboside;
(28) 2-chloro-5′-aminodeoxyadenosine;
(29) 2-fluoro-5′-aminodeoxyadenosine;
(30) 6-methylpurine-5′-aminodeoxyriboside;
(31) 2-amino-5′-aminodeoxyadenosine;
(32) 6-mercaptopurine-5′-aminodeoxyriboside;
(33) 2-chloro-6-methylpurine-5′-aminodeoxyriboside;
(34) 2-chloro-6-mercaptopurine-5′-aminodeoxyriboside;
(35) 5′-aminodeoxyadenosine;
(36) 2-fluoro-6-methylpurine-5′-aminodeoxyriboside;
(37) 2-amino-6-methylpurine-5′-aminodeoxyriboside;
(38) 2-fluoro-6-mercaptopurine-5′-aminodeoxyriboside;
(39) 2-amino-6-mercaptopurine-5′-aminodeoxyriboside;
(40) 6-methylthiopurine-5′-aminodeoxyriboside;
(41) 2-chloro-6-methylthiopurine-5′-aminodeoxyriboside;
(42) 2-fluoro-6-methylthiopurine-5′-aminodeoxyriboside; and
(43) 2-amino-6-methylthiopurine-5′-aminodeoxyriboside
(44) 6-mercaptopurine-5′-deoxyriboside;
(45) 2-fluoro-5′-deoxyadenosine;
(46) 6-methylpurine-5′-deoxyriboside; and
(47) 2-chloro-5′-O-methyladenosine.

More particularly preferred compounds of Formula (I) for treating bacterial or protozoan infections include 2-fluoro-5′-deoxyadenosine, 6-methylpurine-5′-deoxy-β-D-riboside, 2-chloro-5′-O-methyladenosine, 2-chloro-5′-deoxyadenosine, 6-mercaptopurine-5′-deoxy-β-D-riboside, 2-chloro-6-methylpurine-5′-deoxy-β-D-riboside, 2-chloro-6-mercaptopurine-5′-deoxy-β-D-riboside and 2-fluoro-5′-O-methyladenosine.

In another aspect of the invention there is provided compounds of Formula (I), and physiologically acceptable salts and other physiologically functional derivatives thereof, for use in the manufacture of a medicament for the treatment of a bacterial or protozoan infection. In a further aspect of the invention there is provided compounds of Formula (I) for use in inhibiting the growth of a bacteria or protozoa.

It will be appreciated that the compounds of Formula (I) may exist in various tautomeric forms. Compounds of Formula (I) and their salts may also exist in α or β anomeric forms, as well as D- and L-enantiomeric forms. The present invention therefore includes within its scope each of the individual α or β anomeric forms of the compounds of Formula (I), the D- and L-enantiomeric forms of the compounds of Formula (I), combinations thereof, and mixtures thereof.

The compounds of the present invention are particularly effective against those bacteria and protozoa which contain the enzyme adenosine phosphorylase, including, but not limited to, Escherichia coli K-12, Escherichia coli 0157:H7, Shigella flexneri, Salmonella enterica serovar Typhi, Salmonella typhimurium, Yersinia pestis, Klebsiella sp., Pasteurella multocida, Haemophilus influenzae, Actinobacillus pleuropneumoniae, Vibrio cholera, Shewanella oneidensis, Buchnera sp., Helicobacter pylor, Bacillus subtilus, Listeria innocua, Listeria monocytogenes, Lactococcus lactis cremonis, Clostridium peffringens, Enterococcus faecium, Steptococcus pneumoniae, Trichomonas vaginalis, Plasmodium falciparum, Trypanosoma cruzi, Trypanosoma brucei and Leishmania major.

Compounds of Formula (I) can be prepared by a number of methods known in the art, including, but not limited to, methods (A) to (E):

Method (A):

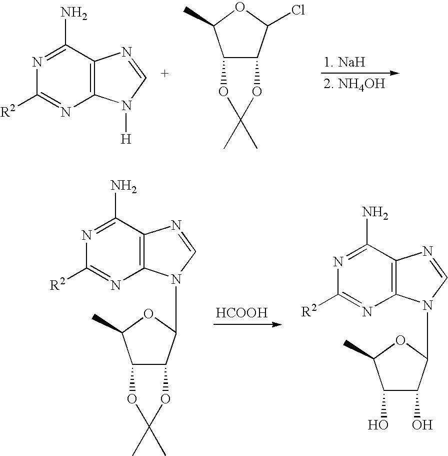

By way of example, method (A) can be used to prepare 2-fluoro-5′-deoxyadenosine (where R² is a fluoro) and 2-amino-5′-deoxyadenosine (where R² is an amino group).

Method (B): 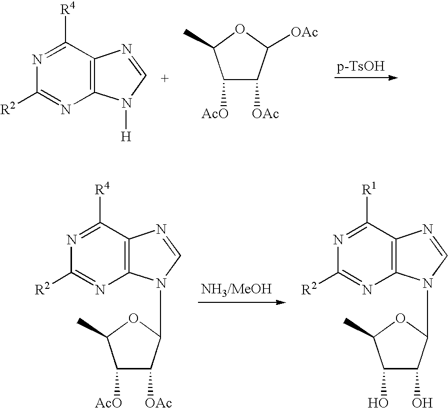
By way of examples, when preparing 2-chloro-5′-deoxyadenosine, R⁴ is a chloro, R² is a chloro and R¹ is an amino, and, when preparing 6-methylpurine-5′-deoxy-β-D-riboside, R⁴ is methyl, R² is a hydrogen and R¹ is methyl.

Method (C): 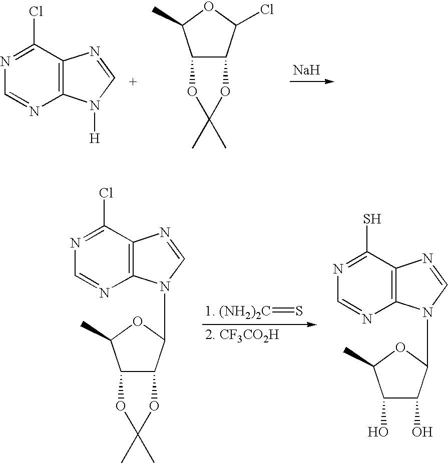
Method C can be used to prepare, for example, 6-mercaptopurine-5′-deoxyriboside.

Method (D): 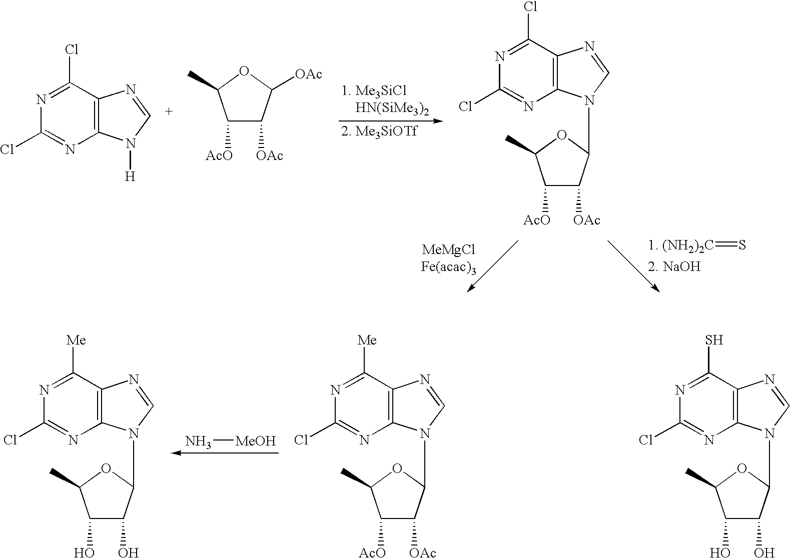
Method D can be used to prepare, for example, 2-chloro-6-methylpurine-5′-deoxy-β-D-riboside and 2-chloro-6-mercaptopurine-5′-deoxy-β-D-riboside.

Method (E): 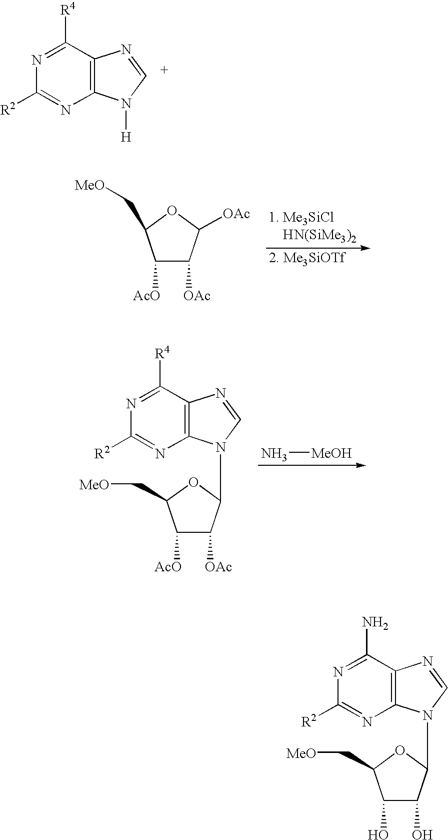
Method (E) can be used to prepare, for example, 2-Chloro-5′-O-methyladenosine, where R⁴ is chloro and R² is chloro, and 2-Fluoro-5′-O-methyladenosine, where R⁴ is amino and R² is fluoro.

According to another aspect of the invention, there is provided a pharmaceutical composition comprising a therapeutically effective amount of a compound of Formula (1). Preferably, the pharmaceutical composition comprises a compound chosen from the preferred compounds; more preferably the compound is chosen from the list of particularly preferred compounds; and most preferably the compound is selected from the group consisting of 2-fluoro-5′-deoxyadenosine, 6-methylpurine-5′-deoxy-β-D-riboside, 2-chloro-5′-O-methyladenosine, 2-chloro-5′-deoxyadenosine, 6-mercaptopurine-5′-deoxy-β-D-riboside, 2-chloro-6-methylpurine-5′-deoxy-β-D-riboside, 2-chloro-6-mercaptopurine-5′-deoxy-β-D-riboside and 2-fluoro-5′-O-methyladenosine.

In another aspect of the invention, there is provided purine nucleoside analogs that are metabolized by bacterial or protozoan AP and not mammalian PNP. The bacterial or protozoan AP catalyzes the conversion of the purine nucleoside analogs to their respective adenine base analogs and these adenine base analogs can further be converted to adenosine monophosphate analogs (AMP^{R}), adenosine diphosphate analogs (ADP^{R}) and ultimately to adenosine triphosphate analogs (ATP^{R}), all of which are toxic to the bacteria or protozoa. Once the adenine analog has been converted to its corresponding nucleotide (AMP^{R}, ADP^{R} or ATP^{R}), these derivatives are effectively trapped within the bacterial or protozoan cell and cannot be taken up by the host cells. By way of example, 2-chloro-5′-deoxyadenosine, 2-fluoro-5′-deoxyadenosine, 6-methyl-5′-deoxyadenosine, 2-chloro-6-methylpurine-5′-deoxyriboside and 2-fluoro-6-methylpurine-5′-deoxyriboside (and their 5′-methoxy, 5′-amino and 2′deoxy equivalents) are converted, respectively, to the toxic base products 2-chloroadenine, 6-methylpurine and 2-chloro-6-methylpurine. Each base product is then converted to their respective toxic nucleotide product(s).

In another aspect, there is provided purine nucleoside analogs that are more refractory to other mammalian enzymes, in particular, adenosine kinase, deoxycytidine kinase, deoxyadenosine kinase and adenosine deaminase. In particular, purine nucleoside analogs have been modified in the 5′-nucleoside position by removing the hydroxyl (—OH) group and adding, for example, a hydrogen, methoxy or amino group in its place. Therefore, these analogs are no longer preferred substrates for direct phosphorylation via adenosine kinase, deoxycytidine kinase or deoxyadenosine kinase. It has been shown that 5′-deoxy-5′-amino-adenosine is a potent inhibitor of adenosine kinase (Kowaluk, E. A., Bhagwat, S. S., and Jarvis, M. C. (1998) Curr Pharm Des 5, 403-416, incorporated herein by reference). Both bacterial and protozoan AP are able to cleave the 5′-deoxyadenosine analogs with comparable rates to that of adenosine or 2′-deoxyadenosine.

In another aspect, there is provided purine nucleoside analogs that have been modified at either the 2-purine position or the 6-purine position or both, and are more refractory to deamination via adenosine deaminase. It has been shown that 2-substituted purines have significantly reduced rates of deamination via adenosine deaminase, a reaction that would generally remove the compound from being a useful pro-drug (Bryson, H. M. and Sorkin. E. M. (1993) Drugs 46, 872-894; Warzocha K., et al (1997) Eur. J. Cancer 33, 170-173). Similarly, 6-substituted purines, other than 6-amino, will also not be suitable substrates for adenosine deaminase. In particular, 6-methylpurine has been shown to be quite refractory to adenosine deaminase.

For use in the present invention, the compound of Formula (I), and physiologically acceptable salts and other physiologically functional derivatives thereof, is preferably presented as a pharmaceutical Formulation. Pharmaceutical Formulations comprise the active ingredient (that is, the compound of Formula (I), and physiologically acceptable salts and other physiologically functional derivatives thereof) together with one or more pharmaceutically acceptable carriers thereof and optionally other therapeutic ingredients. The carrier(s) must be acceptable in the sense of being compatible with the other ingredients of the Formula and not deleterious to the recipient thereof.

Depending on the specific condition or disease state to be treated, subjects may be administered compounds of the present invention at any suitable therapeutically effective and safe dosage, as may be readily determined within the skill of the art. These compounds are, most desirably, administered in dosages ranging from about 1 to about 1000 mg per day, in a single or divided doses, although variations will necessarily occur depending upon the weight and condition of the subject being treated and the particular route of administration chosen. However, a dosage level that is in the range of about 1 to about 250 mg/kg, preferably between about 5 and 100 mg/kg, is most desirable. Variations may nevertheless occur depending upon the weight and conditions of the persons being treated and their individual responses to said medicament, as well as on the type of pharmaceutical Formulation chosen and the time period and interval during which such administration is carried out. In some instances, dosage levels below the lower limit of the aforesaid range may be more than adequate, while in other cases still larger doses may be employed without causing any harmful side effects, provided that such large doses are first divided into several small doses for administration throughout the day.

The compounds of the present invention can be administered in the form of any pharmaceutical Formulation, the nature of which will depend upon the route of administration. These pharmaceutical compositions can be prepared by conventional methods, using compatible, pharmaceutically acceptable excipients or vehicles. Examples of such compositions include capsules, tablets, transdermal patches, lozenges, troches, sprays, syrups, powders, granulates, gels, elixirs, suppositories, and the like, for the preparation of extemporaneous solutions, injectable preparations, rectal, nasal, ocular, vaginal etc.

The preferred route of administration is oral administration. For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tabletting purposes. Solid compositions of similar type may also be employed as fillers in gelatin capsules; preferred materials in this connection also include lactose or milk sugar, as well as high molecular weight polyethylene glycols. When aqueous suspensions and/or elixirs are desired for oral administration the active ingredient may be combined with sweetening or flavoring agents, coloring matter and, if so desired, emulsifying and/or suspending agents, together with such diluents as water, ethanol, propylene glycol, glycerine and various combinations thereof.

The dosage form can be designed for immediate release, controlled release, extended release, delayed release or targeted delayed release. The definitions of these terms are known to those skilled in the art. Furthermore, the dosage form release profile can be effected by a polymeric mixture composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be effected through favorable diffusion, dissolution, erosion, ion-exchange, osmosis or combinations thereof.

For parenteral administration, a solution of an active compound in either sesame or peanut oil or in aqueous propylene glycol can be employed. The aqueous solutions should be suitably buffered (preferably pH greater than 8), if necessary, and the liquid diluent first rendered isotonic. The aqueous solutions are suitable for intravenous injection purposes. The preparation of all these solutions under sterile conditions is readily accomplished by standard pharmaceutical techniques well known to those skilled in the art.

The following non-limitative examples further describe and enable a person ordinarily skilled in the art to make and use the invention.

### DETAILED DESCRIPTION OF THE INVENTION

Bacterial or prozozoan purine nucleoside phosphorylase, or adenosine phosphorylase, catalyzes the reaction: purine nucleoside analog +PO₄→ribose-1-PO₄ (or deoxyribose 1-phosphate)+cytotoxic purine analog. BLAST interrogation of genome databases has confirmed the presence of loci encoding for AP among significant classes of pathogens, as can be seen in Table 1.

TABLE 1
Purine Nucleoside Phosphorylase (BLAST reference E. coli)

| Species | Expect | Identities | Positives | Gaps |
|---|---|---|---|---|
| Escherichia coli K-12 | e−133 | 239/239 (100%) | 239/239 (100%) | 0 |
| Escherichia coli O157:H7 | e−133 | 239/239 (100%) | 239/239 (100%) | 0 |
| Shigella flexneri | e−132 | 238/239 (99%) | 239/239 (100%) | 0 |
| Salmonella enterica serovar Typhi | e−129 | 232/239 (97%) | 235/239 (98%) | 0 |
| Salmonella typhimurium | e−128 | 231/239 (96%) | 235/239 (98%) | 0 |
| Yersinia pestis | e−119 | 216/237 (91%) | 226/237 (95%) | 0 |
| Klebsiella sp. | e−116 | 218/240 (90%) | 224/240 (93%) | 1/240 (0%) |
| Pasteurella multocida | e−111 | 203/235 (86%) | 214/235 (91%) | 0 |
| Haemophilus influenzae | e−109 | 199/235 (84%) | 213/235 (90%) | 0 |
| Actinobacillus pleuropneumoniae | e−106 | 196/238 (82%) | 212/238 (89%) | 0 |
| Vibrio cholerae | e−106 | 191/238 (80%) | 216/238 (90%) | 0 |
| Shewanella oneidensis | 5e−91 | 162/234 (69%) | 197/234 (84%) | 0 |
| Buchnera sp. | 1e−80 | 144/234 (61%) | 187/234 (79%) | 0 |
| Bacillus subtilis | 2e−71 | 134/229 (58%) | 174/229 (75%) | 0 |
| Helicobacter pylori | 2e−69 | 127/229 (55%) | 168/229 (73%) | 0 |
| Listeria innocua | 2e−68 | 132/230 (57%) | 168/230 (73%) | 0 |
| Listeria monocytogenes | 2e−68 | 132/230 (57%) | 168/230 (73%) | 0 |
| Shewanella oneidensis | 8e−68 | 128/232 (55%) | 167/232 (71%) | 0 |
| Lactococcus lactis cremoris | 4e−67 | 133/235 (56%) | 168/235 (71%) | 1/235 (0%) |
| Clostridium perfringens | 7e−64 | 119/230 (51%) | 166/230 (72%) | 0 |
| Enterococcus faecium | 3e−61 | 123/231 (53%) | 162/231 (70%) | 1/231 (0%) |
| Streptococcus pneumoniae | 8e−58 | 116/231 (50%) | 159/231 (68%) | 1/231 (0%) |
| Trichomonas vaginalis | | /236 (57%) | | see Ref. 42 |
| Plasmodium falciparum | 1e−12 | 58/189 (30%) | 92/189 48%) | 9/189 (4%) |

Preparation of Compounds

### EXAMPLE 1

### Preparation of 2-fluoro-5′-deoxyadenosine

1-Chloro-2,3-di-O-isopropylidene-5-deoxy-D-ribofuranose was produced in situ from 2,3-di-O-isopropylidene-5-deoxy-D-ribofuranose (261 mg) by the method of Ugarkar, B. G.; DaRe, J. M.; Kopcho, J. J.; Browne, C. E.; Schanzer, J. M.; Wiesner, J. B. and Erion, M. D. (2000) J. Med. Chem., 43, 2883 and Ugarkar, B. G.; Castellino, A. J.; DaRe, J. M.; 10 Kopcho, J. J.; Wiesner, J. B.; Schanzer, J. M. and Erion, M. D. (2000) J. Med, Chem., 43, 2894. Sodium hydride (60% in mineral oil, 90 mg) was added to a suspension of 2-fluoroadenine (115 mg, obtained from the Aldrich Co.) in dry dimethylformamide (DMF 8 mL), and the mixture was stirred for 2 h. The 1-chloro-2,3-di-O-isopropylidene-5-deoxy-D-ribofuranose, prepared as described above, was added to the reaction mixture containing the 2-fluoroadenine and stirring was continued overnight. The mixture was filtered and the filtrate was concentrated and separated by flash chromatography (ethyl acetate-hexanes, 1:5) to afford a colorless oil. A suspension of the oil in ammonium hydroxide solution (2 mL) was stirred overnight. The solvent was evaporated and the residue was dissolved in 80% formic acid (1 mL). After 4 h, volatile material was removed in vacuum. The residue was purified by flash chromatography (ethyl acetate-methanol, 5:1) to give 2-fluoro-5′-deoxyadenosine as a white solid (6.5 mg), mp 244-245° C., with a ¹H NMR spectrum as reported by Srivastava, P. C. and Robins, R. K. (1977) J. Carbohydrates, Nucleosides and Nucleotides, 4, 93; ¹³C NMR (DMSO-d₆): 158.6 (d, J=203.4 Hz), 157.6 (d, J=20.5 Hz), 150.6 (d, J=20.3 Hz), 140.2, 117.6, 87.8, 79.9, 74.5, 73.0,18.9.

### EXAMPLE 2

### Preparation of 2-chloro-5′-deoxyadenosine

2,6-Dichloropurine was obtained from the Sigma Co. and 1,2,3-tri-O-acetyl-5-deoxy-D-ribose was prepared by the method of Montgomery, J. A. and Hewson, K. (1972) J. Het. Chem. 9, 445. The two compounds were coupled by heating them with a catalytic amount of p-toluenesulfonic acid at 130° C. for 30 min via the procedure of Montgomery, J. A. and Hewson, K. (1972) J. Het. Chem. 9, 445. The resulting 9-(2,3-di-O-acetyl-5-deoxy-β-D-ribofuranosyl)-2,6-dichloropurine was isolated by flash chromatography using a benzene-ethyl acetate gradient as the eluant. This product (100 mg) was heated in methanol saturated with ammonia in a sealed vessel at 100° C. After 18 h, the reaction mixture was concentrated in vacuo and purified by flash chromatography (dichloromethane-methanol, 10:1) to afford 40 mg of 2-chloro-5′-deoxyadenosine: ¹H NMR (CD₃OD) δ 8.20 (s, 1 H), 5.88 (d, J=4.2 Hz, 1 H), 4.68 (m 1 H), 4.09 (m, 2 H), 1.42 (d, J=6.0 Hz, 3 H); ¹³C NMR (CD₃OD) δ 156.7, 154.0, 150.3, 140.0, 118.2, 89.2, 80.4, 74.9, 73.8, 17.7; MS, m/z (%) 285 (M+, 0.6), 182 (51), 134 (100); HRMS calculated for C₁₀H₁₂ClN₅O₃: 285.0629; found: 285.0629.

### EXAMPLE 3

### Preparation of 6-methylpurine-5′-deoxy-β-D-riboside

The general procedure of Montgomery, J. A. and Hewson, K. (1972) J. Het. Chem. 9, 445 was employed. A mixture of 6-methylpurine (0.100 g, obtained from the Sigma Co.) and 1,2,3-tri-O-acetyl-5-deoxy-D-ribose (0.218 g) (as produced in Example 2) was heated at 85° C. for 5 min. p-Toluenesulfonic acid (4 mg) was added and the mixture was heated at 130° C. for 1 h. The cooled reaction mixture was dissolved in benzene, washed with saturated sodium bicarbonate solution, dried and concentrated. The crude product was purified by flash chromatography (ethyl acetate-hexane, 1:4, followed by methanol-ethyl acetate, 2:98) to afford a colorless solid. The latter was dissolved in 2 mL of methanol saturated with ammonia. The solution was left overnight at −5° C. The mixture was evaporated under reduced pressure and purified by flash chromatography (methanol-ethyl acetate, 2:98) to afford 0.107 g of the product as a crystalline solid: mp 160-162° C.; ¹H NMR (CD₃OD) δ 8.79 (s, 1 H), 8.58 (s, 1 H), 6.05 (d, J=4.6 Hz), 4.81 (m, 1 H), 4.14 (m, 2 H), 2.81 (s, 3 H), 1.42 (d, J=5.6 Hz, 3 H); ¹³C NMR (CDCl₃-CD₃OD) δ 159.3, 151.7, 150.1, 143.3, 133.4, 89.7, 80.7, 74.9, 74.2, 18.9, 18.6; MS, m/z (%) 250 (M+, 0.4), 215 (1), 163 (69), 135 (100); HRMS calculated for C₁₁H₁₄N₄O₃: 250.1066; found: 250.1077.

### EXAMPLE4

### Preparation of 2-amino-5′-deoxyadenosine

2,6-Diaminopurine (available from the Sigma Co.) and 1-chloro-2,3-di-O-isopropylidene-5-deoxy-D-ribofuranose (as produced in Example 1) were coupled by the same procedure used in the preparation of 2-fluoro-5′-deoxyadenosine in Example 1 to give 2,3-di-O-isopropylidene-2,6-diaminopurine-5′-deoxy-β-D-riboside. The latter product (51 mg) was stirred in 2 mL of 80% formic acid for 4 h. Volatile material was evaporated and the residue was purified by flash chromatography (ethyl acetate-methanol, 5:1). The product was recrystallized from ethyl acetate to give 17 mg of 2-amino-5′-deoxyadenosine as an off-white solid: mp 135-138° C.; ¹H NMR (DMSO-d₆) δ 7.87 (s, 1 H), 6.65 (s, 2 H), 5.77 (s, 2 H), 5.67 (d, J=4.9 Hz, 1 H), 5.34 (d, J=5.4 Hz, 1 H), 5.03 (d, J=4.6 Hz, 1 H), 4.53 (m, 1 H), 3.90 (m, 2 H), 1.28 (d, J=6.0 Hz, 3 H); ¹³C NMR (DMSO-d₆) δ 160.3, 156.1, 151.8, 135.9, 113.3, 86.8, 79.3, 74.6, 72.9, 19.0; MS, m/z (%) 266 (M+, 16), 179 (14), 150 (100).

### EXAMPLE 5

### Preparation of 6-mercaptopurine-5′-deoxy-β-D-riboside

Sodium hydride (60% in mineral oil, 31 mg) was added to a suspension of 6-chloropurine (119 mg), obtained from the Sigma Co., in dry acetonitrile (20 mL), and the mixture was stirred for 2 h. 1-Chloro-2,3-di-O-isopropylidene-5-deoxy-D-ribofuranose, as produced in Example 1, was added and the mixture was stirred for 5 h. The solvent was evaporated under reduced pressure. Chromatography of the residue (ethyl acetate-hexanes, 1:2, then 2:1) gave 2′,3′-di-O-isopropylidene-6-chloropurine-5′-deoxyriboside as a colorless oil (106 mg). This product (91 mg) and thiourea (67 mg) were refluxed in 2 mL of ethanol for 10 min. The precipitate was filtered and washed with ethanol, yielding 35 mg of a white solid: mp 272-273° C. The latter solid (19 mg) was stirred for 1 h in 0.5 mL of 80% trifluoroacetic acid. The reaction mixture was neutralized with aqueous ammonia, and then concentrated in vacuum and washed thoroughly with ethanol to afford 11 mg of 6-mercaptopurine-5′-deoxy-β-D-riboside as a white powder: mp 203-204° C., with ¹H NMR spectroscopic data in agreement with those reported by Chae, W.-G.; Chan, T. C. K. and Chang, C. (1988), Tetrahedron, 54, 8661; ¹³C NMR (DMSO-d₆) δ 176.1, 145.3, 143.9, 141.6, 135.5, 88.0, 80.2, 74.5, 73.5,18.9.

### EXAMPLE 6

### Preparation of 2-chloro-6-methylpurine-5′-deoxy-β-D-riboside

Trimethylsilyl chloride (11 μL) was added to a suspension of 2,6-dichloropurine (200 mg, obtained from the Sigma Co.) in hexamethyldisilazane (4.2 mL) at 80° C. The clear solution was then heated to 130° C. under argon for 20 h. The reaction mixture was evaporated under reduced pressure. The resulting silylated base (100 mg) and 1,2,3-tri-O-acetyl-5-deoxy-D-ribose (80 mg), produced as in Example 2, were dissolved in dry 1,2-dichloroethane (1 mL) and heated to 80° C. After 5 min trimethylsilyl triflate (19 μL) was added and the mixture was refluxed for 2 h. It was then cooled, diluted with dichloromethane, washed with 5% saturated sodium bicarbonate solution, water and brine. The organic layer was separated, dried and evaporated under reduced pressure. The crude product was purified by flash chromatography (acetone-dichloromethane, 2:98) to afford 54 mg of 2′,3′-di-O-acetyl-2,6-dichloropurine-5′-deoxy-D-riboside. The latter product was converted into 2′,3′-di-O-acetyl-2-chloro-6-methylpurine-5′-deoxy-D-riboside by the general method of Hocek, M. and Dvorakova, H. (2003), J. Org. Chem. 68, 5773. Thus, 2′,3′-di-O-acetyl-2,6-dichloropurine-5′-deoxy-D-riboside (179 mg) and Fe(acac)₃ (20 mg) were dissolved in 2 mL of dry THF. Methylmagnesium chloride in THF solution (3.0 M, 0.47 mmol) was added dropwise under argon with continuous stirring. After 24 h, the reaction was quenched with saturated ammonium chloride solution, extracted repeatedly with chloroform, dried and evaporated. The crude material was purified by flash chromatography (acetone-dichloromethane, 5:95) to afford 107 mg of a colorless oil. The latter product was dissolved in 1 mL of methanol saturated with ammonia. After 24 h at 0° C., the mixture was evaporated under reduced pressure and the crude product was purified by flash chromatography (methanol-dichloromethane, 5:95) to afford 64 mg of 2-chloro-6-methylpurine-5′-deoxy-β-D-riboside as a white solid: mp 179-181° C.; ¹H NMR (DMSO-d₆) δ 8.76 (s, 1 H), 5.90 (d, J=5.1 Hz, 1 H), 4.64 (m, 1 H), 3.99 (m, 2 H), 2.72 (s, 3 H), 1.32 (d, J=6.2 Hz, 3 H); ¹³C NMR (DMSO-d₆) δ 161.1, 152.3, 151.8, 145.2, 132.4, 88.0, 80.4, 74.5, 73.1, 19.1, 18.9; MS, m/z (%) 284 (M+, 0.6), 198 (93), 169 (100); HRMS calculated for C₁₁H₁₃ClN₄O₃: 284.0676; found: 284.0688.

### EXAMPLE 7

### Preparation of 2-chloro-6-mercaptopurine-5′-deoxy-β-D-riboside

2-Chloro-6-mercaptopurine-5′-deoxy-β-D-riboside is prepared from 9-(2,3-di-O-acetyl-5-deoxy-β-D-ribofuranosyl)-2,6-dichloropurine (obtained by the method of Montgomery, J. A. and Hewson, K. (1972) J. Het. Chem. 9, 445), by treatment with thiourea, followed by hydrolysis.

### EXAMPLE 8

### Preparation of 2-Chloro-5′-O-methyladenosine

2,6-Dichloro-9-(2,3-di-O-acetyl-5-O-methyl-β-D-ribofuranosyl)purine was prepared from 2,6-dichloropurine (obtained from the Sigma Co.) and 1,2,3-tri-O-acetyl-5-O-methyl-D-ribose by the method of van Tilburg, E. W.; van der Klein P. A. M.; Kunzel, J. V. F. D.; de Groote, M.; Stannek, C.; Lorenzen, A. and Ijzerman, A. P. (2001) J. Med. Chem., 44, 2966. A solution of the latter product (37 mg) in methanol saturated with ammonia was heated at 100° C. in a sealed vessel for 4 h. The mixture was evaporated under reduced pressure and the crude product was purified by flash chromatography (methanol-ethyl acetate, 2:98) to afford 21 mg of 2-chloro-5′-O-methyladenosine as a white solid: mp 92-94° C., with ¹H NMR spectroscopic data as reported by van Tilburg, E. W.; van der Klein P. A. M.; Kunzel, J. V. F. D.; de Groote, M.; Stannek, C.; Lorenzen, A.; Ijzerman, A. P. (2001) J. Med. Chem., 44, 2966; ¹³C NMR (CD₃OD) δ 158.2, 155.5, 151.9, 141.3, 119.4, 90.3, 85.3, 76.1, 73.4, 72.0, 59.7.

### EXAMPLE 9

### reparation of 2-Fluoro-5′-O-methyladenosine

2-Fluoroadenine (40 mg), obtained from the Sigma Co., was stirred in 4.2 mL of hexamethyidisilazane at 80° C. Trimethylsilyl chloride (11 μL) was added and the solution was heated in a sealed vessel at 130° C. for 20 h. The reaction mixture was then evaporated in vacuum and the residue, along with 1,2,3-tri-O-acetyl-5-O-methyl-D-ribose (76 mg) (prepared by the method of van Tilburg, E. W.; van der Klein P. A. M.; Kunzel, J. V. F. D.; de Groote, M.; Stannek, C.; Lorenzen, A. and Ijzerman, A. P. (2001) J. Med. Chem., 44, 2966) were heated in 1 mL of dichloromethane in a sealed vessel at 80° C. for 5 min. The reaction was cooled and trimethylsilyl triflate (19 μL) was added. The mixture was heated at 40° C. for 2 h. The mixture was then diluted with dichloromethane, washed with 5% sodium bicarbonate solution, dried and evaporated. The crude material was purified by flash chromatography (acetone-dichloromethane, 1:9) to afford 68 mg of 2-fluoro-2′3′-di-O-acetyl-5′-O-methyladenosine, mp 213-215° C. This product (48 mg) was dissolved in a mixture of 2 mL of methanol and 0.5 mL of dichloromethane that had been saturated with ammonia. The mixture was allowed to stand at 0° C. overnight. It was evaporated under reduced pressure to afford 36 mg of 2-fluoro-5′-O-methyladenosine, which was recrystallized from dichloromethane-hexane: mp 231-233° C.; ¹H NMR (CDCl₃-CD₃OD) δ 8.21 (s, 1 H); 5.92 (d, J=4.6 Hz, 1 H), 4.48 (m, 1 H), 4.31 (m, 1 H), 4.18 (m, 1 H), 3.73 (dd, J=10.8, 2.6 Hz, 1 H), 3.63 (dd, J=10.8, 3.8 Hz, 1 H), 3.43 (s, 3 H); ¹³C NMR (DMSO-d₆) δ 158.6 (d, J=202.0 Hz), 157.6 (d, J=20.6 Hz), 150.7 (d, J=20.0 Hz), 139.7, 117.4, 87.4, 83.1, 73.2, 72.3, 70.4, 58.5; MS (ESI), m/z 321.92 (M++Na), 298.18 (M±H).

Enzyme Assays

(a) Adenosine Phosphorylase Assay

Escherichia coli DH5alpha cells were used as the source of adenosine phosphorylase to test the compounds of the present invention. E. coli DH5 alpha cells were harvested in log phase and collected by centrifugation. Cells were lysed by sonication, centrifuged at 10,000×g for 30 min and the supernatant was recovered for assay or storage at −60° C.

Adenosine phosphorylase activity was assayed using the cell free lysate of E. coli as enzyme source for catalysis of the cleavage of nucleoside analogs to their corresponding base analogs in the presence of 50 mM phosphate at pH 7.4. Reaction products were subjected to separation by reverse phase high performance liquid chromatography (HPLC) equipped with continuous scanning diode array detector as described below. Substrates and products were identified by retention time and UV spectra of their peaks.

Samples for HPLC were prepared post reaction by deproteination with 10% v/v of 50% TCA. Following centrifugation at 10,000 g for 5 min the supernatant was recovered for neutralization. A minimal amount of bromophenol blue was added and the sample was titrated with alamine-freon. Following a further centrifugation at 10,000 g for 5 min, the neutralized sample may be stored at −60° C.

Nucleosides and bases in a 10 μl sample were separated on an HPLC equipped with a scanning UV detector from 220 to 320 nm at 5 nm intervals, utilizing a reverse phase Waters Symmetry C18, 4.6×150 mm, 5 um column in tandem with a Waters guard column. Gradient separation was achieved at 30° C. with the mobile phases: A, methanol; C, 10 mM phosphate, pH 3.5; and D, water according to Table 2:

TABLE 2

| Time (min) | A % | C % | D % | Flow rate (mL/min) |
|---|---|---|---|---|
| 0 | 0 | 100 | 0 | 1.0 |
| 5 | 0 | 100 | 0 | 1.0 |
| 30 | 20 | 40 | 40 | 1.0 |
| 35 | 20 | 40 | 40 | 1.0 |
| 45 | 0 | 100 | 0 | 1.0 |

Compounds were identified by retention time and UV spectra at peak height as collected during separation.

The relative rate of conversion of 100 μM nucleoside analog to the corresponding base in 20 min (measured as the % of analog converted) was determined for the following compounds: 5′-deoxyadenosine, 41%; 2-chloroadenosine, 31%; 2-chloro-5′-deoxyadenosine, 8%; 2-fluoro-5′-deoxyadenosine, >80%; 6-thiopurine-5′-deoxyriboside, 61%; 2-amino-5′-deoxyadenosine, 7%. 5′-deoxyadenosine was used as a control to show that analogs having a 5′-deoxy-substition alone are accepted; the product being adenine, which is the natural base. 2-chloroadenosine was used as a control to demonstrate that a modification of adenosine at the 2-position did not alter its ability to act as a substrate for AP.

(b) Mammalian Adenosine Kinase Assay

Adenosine kinase may be assayed under conditions previously described (see, for example, Snyder F F and Lukey T. (1982) Kinetic considerations for the regulation of adenosine and deoxyadenosine metabolism in mouse and human tissues based on a thymocyte model. Biochim Biophys Acta. 696(3):299-307 and Jenuth J P, Mably E R, and Snyder F F. (1996) Modelling of purine nucleoside metabolism during mouse embryonic development:relative routes of adenosine, deoxyadenosine, and deoxyguanosine metabolism. Biochem Cell Biol. 74(2):219-25, incorporated herein by reference) .

Adenosine kinase is assayed using cell lysate from human lymphoblasts. Nucleoside analog, 25-100 μM, 1 mM ATP, 5 mM MgCl2, in 50 mM Tris-HCl, pH 7.4, and cell lysate are incubated at 37° C. of for various times. Reactions are terminated by addition of 1/10 volume of 50% trichloroacetic acid, followed by neutralization with alamine Freon. The 10,000×g supernatants may be analyzed or stored at −60° C. prior to analysis. Reaction products are subjected to anion exchange high performance liquid chromatography (HPLC) for separation of nucleosides and nucleoside 5′-monophosphate products as described below. Substrates and products are monitored by continuous scanning diode array detector and peaks are identified in comparison to standards, retention time and UV spectrum.

Samples are prepared for HPLC post reaction by deproteination with 10% v/v of 50% TCA. Following centrifugation at 10,000 g for 5 min the supernatant is recovered. A minimal amount of bromophenol blue is added and the sample is neutralized by titration with alamine-freon. Following a further centrifugation at 10,000 g for 5 min, the neutralized sample may be stored at −60° C.

Nucleoside-′5-monophosphates, -diphosphates and -triphosphates in a 10 μl sample are separated on an HPLC equipped with a scanning UV detector utilizing an anion exchange, Whatman Partisphere 5 SAX, 5 um, 5.6×250 mm column, in tandem with a Whatman anion exchange Guard cartridge. Separation of nucleotides is achieved at 30° C. with a gradient formed from the mobile phases: B, 1M phosphate, pH 3.5; C, 10 mM phosphate, pH 3.5 according to Table 3.

TABLE 3

| Time (min) | B % | C % | Flow rate (mL/min) |
|---|---|---|---|
| 0 | 0 | 100 | 1.0 |
| 5 | 0 | 100 | 1.0 |
| 25 | 70 | 30 | 1.0 |
| 40 | 70 | 30 | 1.0 |
| 50 | 0 | 100 | 1.0 |

Compounds are identified by retention time and UV spectra at peak height as collected during separation.
(c) Mammalian Deoxycytidine/Deoxyadenosine Kinase Assay

Deoxycytidine kinase may be assayed under conditions previously described (see, for example, Snyder F F, Jenuth J P, Dilay J E, Fung E, Lightfoot T, and Mably E R. (1994) Secondary loss of deoxyguanosine kinase activity in purine nucleoside phosphorylase deficient mice. Biochim Biophys Acta. 1227(1-2): 33-40, incorporated herein by reference).

Mammalian deoxyadenosine kinase may be assessed under conditions previously described (see, for example, Jenuth J P, Mably E R, and Snyder F F. (1996) Modelling of purine nucleoside metabolism during mouse embryonic development: relative routes of adenosine, deoxyadenosine, and deoxyguanosine metabolism. Biochem Cell Biol. 74(2): 219-25 and Snyder F F, Jenuth J P, Dilay J E, Fung E, Lightfoot T, and Mably E R. (1994) Secondary loss of deoxyguanosine kinase activity in purine nucleoside phosphorylase deficient mice. Biochim Biophys Acta. 1227(1-2): 33-40, incorporated herein by reference).

2′-deoxynucleoside analogs may be phosphorylated by an individual or a combination of deoxyribonucleoside kinases, which for 2′-deoxyadenosine analogs principally include deoxycytidine kinase and deoxyadenosine kinase activities. The assay utilizes a cell free cytoplasmic supernatant from a human lymphoblast. Cell extract plus deoxyribonucleoside analogs, 25-200 μM, 1 mM ATP, 5 mM MgCl2, are incubated at 37° C. in 50 mM Tris-HCl, pH 7.4 for various periods of time. Reactions are terminated by addition of 1/10 volume of 50% trichloroacetic acid followed by neutralization with alamine Freon. The 10,000×g supernatants may be stored at −60° C. prior to analysis. Reaction products are subjected to anion exchange high performance liquid chromatography for separation of nucleosides and nucleoside 5′-monophosphate products as described in (b) for the mammalian adenosine kinase assay. Substrates and products are monitored by continuous scanning diode array detector and peaks are identified in comparison to standards, retention time and UV spectrum.

(d) Mammalian Adenosine Deaminase Assay

Adenosine deaminase may be assayed under conditions previously described (see, for example, Snyder F F, and Lukey T. (1982) Kinetic considerations for the regulation of adenosine and deoxyadenosine metabolism in mouse and human tissues based on a thymocyte model. Biochim Biophys Acta. 696(3): 299-307, incorporated herein by reference).

Because bacterial and mammalian adenosine deaminase activities have similar specificities, the assays for deaminase activity were conducted at the same time as the adenosine phosphorylase assays using E. coli lysates. As previously described, E. coli DH5alpha cells were harvested in log phase and collected by centrifugation. Cells were lysed by sonication, centrifuged at 10,000×g for 30 min and the supernatant was recovered for assay or storage at −60° C.

Adenosine deaminase activity was assayed using the cell free lysate of E. coli as enzyme source for catalysis of the deamination of nucleoside analogs, 100 μM, to their corresponding base analogs in the presence of 50 mM phosphate at pH 7.4 at 37° C. Reaction products were subjected to separation by reverse phase high performance liquid chromatography (HPLC) equipped with continuous scanning diode array detector as described below. Substrates and products are identified by retention time and UV spectra of their peaks. No deamination products were observed for any of the compounds tested.

Antibacterial Activity

Analogs were examined for their ability to inhibit the growth of E. coli DH5alpha cultures in log phase by monitoring the cell density at 600 nm at various times over a 250 minute time course. The relative growth inhibition for several nucleoside analogs is given in Table 4.

TABLE 4

| | Relative growth inhibition | | | | |
|---|---|---|---|---|---|
| Nucleoside analog (μM) | 3.1 | 10 | 31.3 | 100 | 200 |
| 2-fluoro-5′-deoxyadenosine | − | + | ++ | +++ | ++++ |
| 2-chloro-5′-deoxyadenosine | − | − | + | ++ | +++ |
| 6-thiopurine-5′-deoxyadenosine | | | | + | |
| 2-amino-9-(beta-D-ribofuranosyl)-purine | | | | | ++ |

Demonstration of Safety and Efficacy

The desired metabolic properties of the analogs of Formula (I) are optimized by utilizing enzyme preparations from pathogen and human cell line lysates and recombinant enzymes expressed and purified from pathogen sources.

Specific bacterial strains and protozoan targets are used for analysis of their capability to activate the nucleoside and base analogs. Lead analogs having a significant rate of transformation are further studied in comparative toxicity assays against bacterial and protozoan cultures versus human cell lines. In vitro screens of analog efficacy use bacterial strains of Streptococcus, Pseudomonas and Staphylococcus, and the protozoa Cryptosporidium and Giardia.

Baseline toxicity studies are conducted in mice, as a prelude to assessment of analog effectiveness in pathogen infected animal models. In vivo models, such as that for pulmonary infection by Pseudomonas aeruginosa and gastrointestinal infection by protozoa, are used to establish both safety and efficacy.

1. A compound of the general Formula: 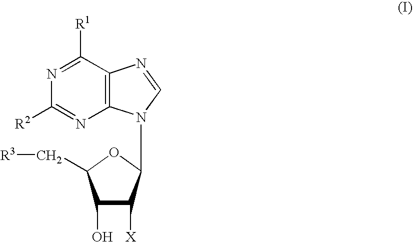
wherein:
R¹ is an amino, lower alkyl, sulfhydryl, lower alkylthio or lower alkoxy;
R² is a halogen, amino, hydrogen or lower alkyl;
R³ is an amino, lower alkoxy, lower alkyl or hydrogen; and
X is a hydroxy or hydrogen;
provided that:
(a) when X is hydroxy, R² is fluoro and R¹ is amino, R³ is not hydrogen;
(b) when X is hydroxy and R¹ is methyl, R² and R³ are not both hydrogen;
(c) when X is hydroxy, R² is chloro and R³ is methoxy, R¹ is not amino; and
(d) when X is hydroxy, R¹ is sulfhydryl and R² is hydrogen, R³ is not hydrogen;
or a tautomer thereof; or a physiologically acceptable salt or solvates thereof; or a prodrug thereof.

2. The compound of claim 1 wherein:
R¹ is an amino, methyl, sulfhydryl or methylthio group;
R² is chloro, fluoro, amino group or hydrogen; and
R³ is hydrogen, methoxy or amino group;
provided that:
(a) when X is hydroxy, R² is fluoro and R¹ is amino, R³ is not hydrogen;
(b) when X is hydroxy and R¹ is methyl, R² and R³ are not both hydrogen;
(c) when X is hydroxy, R² is chloro and R³ is methoxy, R¹ is not amino; and
(d) when X is hydroxy, R¹ is sulflhydryl and R² is hydrogen, R³ is not hydrogen;
or a tautomer thereof; or a physiologically acceptable salt or solvates thereof; or a prodrug thereof.

3. The compound of claim 1 selected from the group consisting of:
2-chloro-5′-deoxyadenosine;
2-chloro-6-methylpurine-5′-deoxyriboside;
2-chloro-6-mercaptopurine-5′-deoxyriboside;
5′-deoxyadenosine;
2-fluoro-6-methylpurine-5′-deoxyriboside;
2-amino-6-methylpurine-5-deoxyriboside;
2-fluoro-6-mercaptopurine-5′-deoxyriboside;
2-amino-6-mercaptopurine-5′-deoxyriboside;
6-methylthiopurine-5′-deoxyriboside;
2-chloro-6-methylthiopurine-5′-deoxyriboside;
2-fluoro-6-methylthiopurine-5′-deoxyriboside;
2-amino-6-methylthiopurine-5′-deoxyriboside;
2-fluoro-5′-O-methyladenosine;
6-methylpurine-5′-O-methylriboside;
2-amino-5′-O-methyladenosine;
6-mercaptopurine-5′-O-methylriboside;
2-chloro-6-methylpurine-5′-O-methylriboside;
2-chloro-6-mercaptopurine-5′-O-methylriboside;
5′-O-methyladenosine;
2-fluoro-6-methylpurine-5′-O-methylriboside;
2-amino-6-methylpurine-5′-O-methylriboside;
2-fluoro-6-mercaptopurine-5′-O-methylriboside;
2-amino-6-mercaptopurine-5′-O-methylriboside;
6-methylthiopurine-5′-O-methylriboside;
2-chloro-6-methylthiopurine-5′-O-methylriboside;
2-fluoro-6-methylthiopurine-5′-O-methylriboside;
2-amino-6-methylthiopurine-5′-O-methylriboside;
2-chloro-5′-aminodeoxyadenosine;
2-fluoro-5′-aminodeoxyadenosine;
6-methylpurine-5′-aminodeoxyriboside;
2-amino-5′-aminodeoxyadenosine;
6-mercaptopurine-5′-aminodeoxyriboside;
2-chloro-6-methylpurine-5′-aminodeoxyriboside;
2-chloro-6-mercaptopurine-5′-aminodeoxyriboside;
5′-aminodeoxyadenosine;
2-fluoro-6-methylpurine-5′-aminodeoxyriboside;
2-amino-6-methylpurine-5′-aminodeoxyriboside;
2-fluoro-6-mercaptopurine-5′-aminodeoxyriboside;
2-amino-6-mercaptopurine-5′-aminodeoxyriboside;
6-methylthiopurine-5′-aminodeoxyriboside;
2-chloro-6-methylthiopurine-5′-aminodeoxyriboside;
2-fluoro-6-methylthiopurine-5′-aminodeoxyriboside; and
2-amino-6-methylthiopurine-5′-aminodeoxyriboside;
or a tautomer thereof; or a physiologically acceptable salt or solvate thereof; or a prodrug thereof.

4. The compound of claim 1 selected from the group consisting of 2-chloro-5′-deoxyadenosine, 2-chloro-6-methylpurine-5′-deoxy-β-D-riboside, 2-chloro-6-mercaptopurine-5′-deoxy-β-D-riboside and 2-fluoro-5′-O-methyladenosine, or a tautomer thereof, or a physiologically acceptable salt-, or solvate thereof; or a prodrug thereof.

5. A pharmaceutical composition for the treatment of a bacterial or protozoan infection comprising a therapeutically effective amount of a compound of claim 1, and a pharmaceutically acceptable carrier or diluent.

6. A method of treating a bacterial or protozoan infection in a mammal in need thereof which comprises administering to said mammal a therapeutically effective amount of a compound of the general Formula: 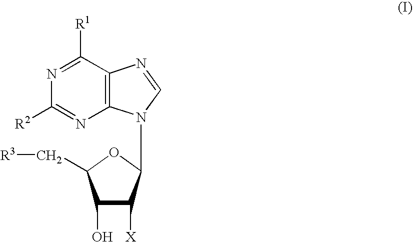
wherein:
R¹ is an amino, lower alkyl, sulfhydryl, lower alkylthio, or lower alkoxy;
R² is a halogen, amino, hydrogen or lower alkyl;
R³ is an amino, lower alkoxy, lower alkyl or hydrogen; and
X is a hydroxy or hydrogen;
or a tautomer thereof; or a physiologically acceptable salt or solvates thereof; or a prodrug thereof.

7. The method of claim 6 wherein:
R¹ is an amino, methyl, sulfhydryl or methylthio group;
R² is chloro, fluoro, amino group or hydrogen;
R³ is hydrogen, methoxy or amino group; and
X is a hydroxy or hydrogen;
or a tautomer thereof; or a physiologically acceptable salt or solvate thereof; or a prodrug thereof.

8. The method of claim 6 wherein the compound is selected from the group consisting of:
2-chloro-5′-deoxyadenosine;
6-mercaptopurine-5′-deoxyriboside;
2-chloro-6-methylpurine-5′-deoxyriboside;
2-chloro-6-mercaptopurine-5′-deoxyriboside;
5′-deoxyadenosine;
2-fluoro-6-methylpurine-5′-deoxyriboside;
2-amino-6-methylpurine-5′-deoxyriboside;
2-fluoro-6-mercaptopurine-5′-deoxyriboside;
2-amino-6-mercaptopurine-5-′deoxyriboside;
6-methylthiopurine-5′-deoxyriboside;
2-chloro-6-methylthiopurine-5′-deoxyriboside;
2-fluoro-6-methylthiopurine-5′-deoxyriboside;
2-amino-6-methylthiopurine-5′-deoxyriboside;
2-fluoro-5′-O-methyladenosine;
6-methylpurine-5′-O-methylriboside;
2-amino-5′-O-methyladenosine;
6-mercaptopurine-5′-O-methylriboside;
2-chloro-6-methylpurine-5′-O-methylriboside;
2-chloro-6-mercaptopurine-5′-O-methylriboside;
5′-O-methyl adenosine;
2-fluoro-6-methylpurine-5′-O-methylriboside;
2-amino-6-methylpurine-5′-O-methylriboside;
2-fluoro-6-mercaptopurine-5′-O-methylriboside;
2-amino-6-mercaptopurine-5′-O-methylriboside;
6-methylthiopurine-5′-O-methylriboside;
2-chloro-6-methylthiopurine-5′-O-methylriboside;
2-fluoro-6-methylthiopurine-5′-O-methylriboside;
2-amino-6-methylthiopurine-5′-O-methylriboside;
2-chloro-5′-aminodeoxyadenosine;
2-fluoro-5′-aminodeoxyadenosine;
6-methylpurine-5′-aminodeoxyriboside;
2-amino-5′-aminodeoxyadenosine;
6-mercaptopurine-5′-aminodeoxyriboside;
2-chloro-6-methylpurine-5′-aminodeoxyriboside;
2-chloro-6-mercaptopurine-5′-aminodeoxyriboside;
5′-aminodeoxyadenosine;
2-fluoro-6-methylpurine-5′-aminodeoxyriboside;
2-amino-6-methylpurine-5′-aminodeoxyriboside;
2-fluoro-6-mercaptopurine-5′-aminodeoxyriboside;
2-amino-6-mercaptopurine-5′-aminodeoxyriboside;
6-methylthiopurine-5′-aminodeoxyriboside;
2-chloro-6-methylthiopurine-5′-aminodeoxyriboside;
2-fluoro-6-methylthiopurine-5′-aminodeoxyriboside;
2-amino-6-methylthiopurine-5′-aminodeoxyriboside;
2-fluoro-5′-deoxyadenosine;
6-methylpurine-5′-deoxyriboside; and
2-chloro-5′-O-methyladenosine;
or a tautomer thereof; or a physiologically acceptable salt or solvates thereof; or a prodrug thereof.

9. The method of claim 6 wherein the compound is selected from the group consisting of 2-fluoro-5′-deoxyadenosine, 6-methylpurine-5′-deoxy-β-D-riboside, 2-chloro-5′-O-methyladenosine, 2-chloro-5′-deoxyadenosine, 6-mercaptopurine-5′-deoxy-β-D-riboside, 2-chloro-6-methylpurine-5′-deoxy-β-D-riboside, 2-chloro-6-mercaptopurine-5′-deoxy-β-D-riboside and 2-fluoro-5′-O-methyladenosine, or a tautomer thereof, or a physiologically acceptable salt or solvate thereof; or a prodrug thereof.

10. The method of claim 6, wherein the bacteria or protozoa causing the infection is selected from the group consisting of Escherichia coli K-12, Escherichia coli 0157:H7, Shigella flexneri, Salmonella enterica serovar Typhi, Salmonella typhimurium, Yersinia pestis, Klebsiella sp., Pasteurella multocida, Haemophilus influenzae, Actinobacillus pleuropneumoniae, Vibrio cholera, Shewanella oneidensis, Buchnera sp., Helicobacter pylori, Bacillus subtilus, Listeria innocua, Listeria monocytogenes, Lactococcus lactis cremonis, Clostridium perfringens, Enterococcus faecium, Steptococcus pneumoniae, Trichomonas vaginalis, Plasmodium falciparum, Trypanosoma cruzi, Trypanosoma brucei and Leishmania major.

11. The method of claim 10 wherein the bacteria causing the infection is Escherichia coli.

12. (canceled)

13. (canceled)

14. (canceled)

15. (canceled)

16. (canceled)

17. (canceled)

18. (canceled)
